(19) United States
(12) Patent Application Publication
(10) Pub. No.: US 2010/0298223 A1
(43) Pub. Date: Nov. 25, 2010

(54) FIBRINOGEN FOR TREATMENT OF BLEEDING IN TRAUMA AND PLATELET DISORDERS

(76) Inventors: Dietmar Rudolf Fries, Innsbruck (AT); Uri Martinowitz, Ramat Efal (IL)

Correspondence Address: FENNEMORE CRAIG, 3003 NORTH CENTRAL AVENUE, SUITE 2600, PHOENIX, AZ, 85012, US

(21) Appl. No.: 12/598,562

(22) PCT Filed: Feb. 28, 2008
(86) PCT No.: PCT/IL08/00254

§ 371 Date: Jun. 17, 2010

Related U.S. Application Data
(63) Continuation-in-part of application No. PCT/IL2007/001076, filed on Aug. 30, 2007.
(60) Provisional application No. 60/915,548, filed on May 2, 2007.

(51) Int. Cl. A61K 38/36; A61P 7/02

(52) U.S. Cl. 514/13.7

(57) ABSTRACT

The present invention relates generally to use of fibrinogen to prevent or treat excessive bleeding in pre-hospital and hospital settings. In particular, the present invention relates to methods for treating bleeding using fibrinogen in individuals suffering from traumatic hemorrhages in pre-hospital settings and in individuals having thrombocytopenia or qualitative platelet disorders.

### FIELD OF THE INVENTION

The present invention relates generally to use of fibrinogen to prevent or treat excessive bleeding in pre-hospital and hospital settings. In particular, the present invention relates to methods for treating bleeding using fibrinogen in individuals suffering from traumatic hemorrhages in pre-hospital settings and in individuals having thrombocytopenia or qualitative platelet disorders.

### BACKGROUND OF THE INVENTION

Hemorrhage is the most common cause of death among trauma patients and is the leading cause of death of young people including those who die prior to reaching care, who die in emergency medical care or who die in the operating room. The most common causes of death of individuals in post-operative critical care are those involving sequellae of poorly controlled hemorrhage and shock. In the prehospital setting, most internal bleeding is not accessible for direct hemostasis. Even in the hospital setting, there are sources of bleeding which cannot be controlled even with the best surgical techniques.

Fibrin has been used as a local hemostatic agent as early as 1909 when wound scabs were dried, powdered, and sprinkled on wounds. Later on, two component fibrin glues, based on fibrinogen and thrombin were developed and became widespread. (Tock et al., Hemophilia 4: 449-455, 1998; Martinowitz and Schulman, Haemostas. 74(1): 486-492, 1995). Fibrinogen was isolated from human plasma in bulk quantities by Cohn during World War II, and was used for both fibrin glues and fibrinogen concentrates for infusion. The use of fibrinogen was stopped due to transmission of hepatitis virus and other blood borne infectious agents. The FDA removed the last human fibrinogen products from the market on Dec. 31, 1977. As a result, clinicians switched to using single donor blood products, fresh frozen plasma and cryoprecipitate as sources of injectable fibrinogen to treat severe bleeding. However, the disadvantage of the use of such fresh frozen products or cryoprecipitate is the need to maintain these products in a frozen state, making them less available and convenient for emergency use in the hospital and unsuitable for use in pre-hospital settings. In addition, although these products are screened for various blood borne infectious agents, they are still unsafe and may transmit viral, bacterial and other blood borne transmissible agents. In addition they may cause serious immunological complications.

### Fibrinogen

The human fibrinogen protein ordinarily circulates in the plasma at concentrations of 2-4 g/L plasma. In the uncleaved form, fibrinogen is inert in the bloodstream, and the activated form is not normally found in healthy blood vessels. Activation by thrombin occurs by cleavage of small activation peptides from the free ends of the paired alpha and beta chains. This converts fibrinogen to fibrin monomer by exposing “sticky” ends on the fibrin monomeric units. The polymer can be formed by building a matrix from fibrin monomers. Fibrin binds to collagen and receptors on platelets, anchoring it to tissue in wounds and the other components of the clot. Adherent clot begins to form at the edge of the wound and builds a mass of adherent clot, which grows toward the center of the wound, recruiting platelets which form the basis for new activation sites.

Because fibrin is a structural protein, its effect is a direct function of the amount present. A correlation between fibrinogen content and clot strength has been reported. The concentration of fibrin in injured vessels is a direct function of the rate of production from fibrinogen and the rate of loss through the wound or from breakdown. The rate of fibrin production is a function of thrombin enzyme activity and of the concentration of the fibrinogen substrate. Thus, when a wound is formed that breaks a blood vessel, blood is exposed to tissues which activate the coagulation system. Activation starts the clotting at the edge of the wound and builds a mass of adherent clot which grows toward the center of the wound by recruiting platelets which form the basis for new activation sites and tying them to the wound edge with fibrin. As long as the strength of the clot and its adherence to the wound edge are greater than the other forces operating on the wound such as blood pressure, the clot continues to grow and progressively occludes and seals the wound. This basic coagulation mechanism works well in small wounds, poorly in large arterial disruptions, and performs variably for wounds of intermediate severity.

U.S. Pat. No. 6,825,323 to Hess discloses compositions of factor VIIa and fibrinogen and methods of using these compositions to minimize or stop traumatic bleeding at internal and/or external wound sites by activating the blood clotting system at sites of injury. U.S. Pat. No. 6,825,323 explicitly requires the combination of factor VIIa and fibrinogen to treat mild to severe bleeding due to open wounds, liver hemorrhaging, bleeding disorders and blood clotting disorders.

U.S. Pat. No. 7,045,601 discloses a storage-stable, liquid or viscous liquid fibrinogen formulation comprising fibrinogen, divalent metal ions and a complexing agent, wherein the fibrinogen formulation is stable at storage temperatures between 0° C. and 30° C. for at least 1 month.

### Thrombocytopenia

Platelet disorders can be divided into disorders of platelet function or number. Thrombocytopenia is defined as a platelet count less than 150,000/mm³. It can be caused by decreased platelet production, increased destruction, sequestration, or a combination of these causes. With normal platelet function, thrombocytopenia is rarely the cause of bleeding unless the count is less than 50,000/mm³.

Thrombocytopenia is a complication frequently observed in hospitalized patients, resulting from primary or secondary impaired platelet production, accelerated platelet destruction, either immune (e.g., as ITP—idiopathic thrombocytopenic purpura) or non-immune (e.g., as destruction by artificial surfaces), increased consumption at injury sites or due to disseminated intravascular coagulation (DIC), or excessive dilution as in massive transfusion. Platelet transfusion is indisputably indicated in bleeding patients with counts below 50×10⁹/L, but between 50×10⁹/L and 100×10⁹/L the recommendations are vague. The therapeutic effect of transfused platelets cannot be predicted with certainty. Storage, platelet age and the recipient's condition alter the efficacy of platelet transfusion. The transfusion of platelet concentrate is potentially hazardous and the indication should therefore be strict. Complications include transfusion-related lung injury (TRALI) or, more frequently, severe bacterial infection and even sepsis as well as transfusion related transmission of viruses. In addition, the short storage life of five days limits their availability and many remote hospitals do not keep platelet concentrates. For this reason, platelets were not available in the first two years of the Iraq war and the US Army used fresh whole blood as platelets substitute, until they introduced plateletpheresis. It also restricts their use to in-hospital settings only.

Low platelet count primarily affects clot firmness, which can be easily monitored by thrombelastography. Clot firmness is also influenced by fibrinogen and coagulation factor XIII (Fries et al., Br. J. Anaesth. 95: 172-177, 2005; Lorand et al., J. Thromb. Haemost. 3: 1337-1348, 2005; Nielsen et al., Anaesth. Analg. 99: 120-123, 1999). In cardiac surgery, most bleedings are due to quantitative or qualitative platelets disorders (thrombocytopenia and thrombocytopathy) caused by platelet activation and destruction due to extracorporeal surfaces. However, it has been observed that patients with high fibrinogen values experienced fewer bleeding complications than patients with low fibrinogen values (Fries et al., Anaesth. Analg. 99: 947, 2004; Biome et al., Thromb. Haemost. 93: 1101-1107, 2005; Pothula et al., Anaesth. Analg. 98: 4-10, 2004). Fibrinogen plays an important role in the coagulation process and clot stabilization binding of factor XIII. In addition, it plays a central role in platelet activation and aggregation by binding to the platelet glycoprotein receptor GPIIb/IIIa. It has been shown that the effect of platelet-blocking substances like clopidogrel can be antagonized by increasing the fibrinogen concentration.

### Qualitative Platelet Disorders

Qualitative platelet disorders are suggested by a prolonged bleeding time (abnormal platelet function screen) or clinical evidence of bleeding in the setting of a normal platelet count and coagulation studies. Qualitative platelet disorders are most commonly acquired, but can be inherited.

Inherited platelet disorders include Glanzmann's Thrombasthenia and Bernard-Soulier disease. Glanzmann's Thrombasthenia (GT) is a rare congenital bleeding disorder caused by deficiency or dysfunction of platelet surface glycoprotein (GP) IIb/IIIa receptor. Platelet transfusion is the standard treatment for bleeding that remains non-responsive to conservative measures, and for surgical coverage. Platelet transfusions, however, may result in the development of antibodies to GPIIb/IIIa and/or to human leukocyte antigen (HLA), rendering further transfusions ineffective. Recombinant human activated factor VII (rFVIIa) has been documented as efficient in GT patients, and is approved in Europe and in the U.S. for the treatment of GT patients. However, the response to rFVIIa is unpredictable and disappointing and of short duration (half life of 2 hours). Patients may require frequent repeated doses, and treatment of bleeding episodes or surgery may be extremely expensive.

There is still an unmet need for improved methods of treating bleeding in individuals suffering from traumatic hemorrhaging in pre-hospital settings as well as in individuals having thrombocytopenia or qualitative platelet disorders, which methods can minimize the necessity for platelet or blood transfusion and improve survival.

### SUMMARY OF THE INVENTION

The present invention provides methods of treating an individual suffering from excessive bleeding in a pre-hospital setting comprising administering to the individual human fibrinogen in order to improve clot quality and achieve hemostasis. The present invention further provides methods of treating bleeding in individual suffering from thrombocytopenia comprising administering to the individual human fibrinogen. The present invention yet further provides methods of treating bleeding in an individual suffering from a qualitative platelet disorder comprising administering to the individual human fibrinogen. The present invention still further provides methods of treating excessive bleeding in an individual having plasma fibrinogen within the normal or physiological range comprising administering to the individual human fibrinogen. The fibrinogen useful in the methods of the present invention is virally inactivated fibrinogen concentrate isolated or purified from human plasma substantially devoid of additional blood components or recombinant human fibrinogen.

Previous and current medical practice has recommended administration of fibrinogen in bleeding patients when fibrinogen levels in plasma are below 1 g/L or sometimes even below 1.5 g/L. Administration of fibrinogen in such cases is considered as “replacement” therapy.

It is now disclosed that administration of human fibrinogen to individuals suffering from excessive bleeding due to trauma or surgery is effective to stop the bleeding when the plasma level of fibrinogen is above 1-1.5 g/L and even when it is within the normal range (i.e., 2-4 g/L). The present invention demonstrates that exogenously added fibrinogen, in the absence of added factor VIIa or any other coagulation factors, is capable of improving clot formation and clot firmness and thus reduces the need for administration of other coagulation factors and/or transfusion of platelets.

The methods of the present invention are particularly useful in cases of individuals suffering from excessive bleeding in pre-hospital settings where blood or platelet transfusion is not available while the survival of the individuals in endangered due to massive blood loss. It should be appreciated that the common standard of care of individuals suffering from excessive bleeding in pre-hospital settings is very limited and involves pressure on the wounds or application of tourniquets, treatment with haemostatic bandages, and infusion of fluids to compensate for blood volume loss. Nowhere in the background art is it taught that haemostatic agents, particularly fibrinogen, can be used to treat or prevent excessive bleeding in pre-hospital settings.

The present invention discloses for the first time that intravenous administration of human fibrinogen to individuals suffering from excessive bleeding in pre-hospital settings can save lives in such trauma cases. In cases where infusion of fluids is necessary to compensate for blood volume loss, such infusion should be performed after fibrinogen administration so that fibrinogen strengthens the clot before hemodilution. Thus, according to the principles of the present invention, the haemostatic effect of the exogenously added fibrinogen is greatly improved when neither the exogenously added fibrinogen nor the endogenous coagulation factors and platelets are diluted by large volumes of fluids commonly infused to compensate for blood volume loss. The methods of the present invention achieve fast and efficient arrest of uncontrolled bleeding even before coagulopathy develops, diminish blood loss and reduce the need for blood and/or platelet transfusion. Moreover, as pharmaceutical compositions comprising human fibrinogen can be prepared as stable-storage compositions, even at ambient temperatures, such compositions are particularly useful for treating bleeding in pre-hospital settings.

It is now further disclosed that administration of fibrinogen concentrate is therapeutically beneficial in overcoming impaired clot formation and increased bleeding in severe thrombocytopenia. As exemplified herein below in animal studies using a porcine model of thrombocytopenia with uncontrolled hemorrhage as well as in thrombocytopenic bleeding humans the administration of fibrinogen concentrate improved clot formation and decreased bleeding in the treated animals and humans. The present invention teaches that the functional consequences of thrombocytopenia (decreased clot firmness, increased bleeding) can be at least partially overcome by administering fibrinogen concentrate. It is to be understood that the methods of the present invention both minimize the risk of introducing detrimental foreign agents as well as economize the therapeutic benefit by decreasing or replacing the need for platelet transfusion.

According to one aspect, the present invention provides a method for treating a subject suffering from excessive bleeding in a pre-hospital setting comprising administering to the subject an anti-hemorrhagic pharmaceutical composition consisting of human fibrinogen as the active ingredient, wherein administration of the pharmaceutical composition is performed in a pre-hospital setting. According to a certain embodiment, administration of the anti-hemorrhagic pharmaceutical composition is performed prior to infusion of fluids which compensate for blood volume loss. According to a particular embodiment, administration of the anti-hemorrhagic pharmaceutical composition is performed concomitantly with infusion of fluids, wherein the volume of the fluids is lower than about 500 ml, preferably lower than about 250 ml. According to another particular embodiment, administration of the anti-hemorrhagic pharmaceutical composition is performed shortly after initiation of infusion of fluids, wherein the volume of the fluids is lower than about 500 ml, preferably lower than about 250 ml.

According to some embodiments, human fibrinogen is selected from the group consisting of fibrinogen isolated from human plasma, and recombinant human fibrinogen. It is to be appreciated that use of recombinant fibrinogen should minimize the risk of introducing detrimental foreign agents. According to a certain embodiment, human fibrinogen is human fibrinogen concentrate which is virus free and/or has undergone a plurality of viral inactivation steps and is devoid of other blood components.

According to additional embodiments, the excessive bleeding is due to any variety of causes including, but not limited to, traumatic injury, open wounds, and internal bleeding such as in liver injury.

According to further embodiments, the pharmaceutical composition is administered by intravenous injection or infusion. It is to be appreciated that when the anti-hemorrhagic pharmaceutical composition is administered by injection or infusion, the volume of the pharmaceutical composition is up to 100 ml, alternatively up to 50 ml, further alternatively up to 30 ml, yet further alternatively up to 10 ml. According to a certain embodiment, the pharmaceutical composition is administered by intravenous bolus injection in a volume of up to 50 ml.

According to the principles of the present invention human fibrinogen is administered in an amount effective to cause hemostasis. According to some embodiments, human fibrinogen is present within the pharmaceutical composition in an amount ranging from about 1 g to about 15 g, alternatively from about 2 g to about 10 g, further alternatively from about 2 g to about 5 g, yet further alternatively at about 4 g. The dosage of human fibrinogen to be administered will be determined by the severity of bleeding, the weight and clinical situation of the individual, and the like. Human fibrinogen can be administered in a single dose or multiple times in order to decrease or stop bleeding. According to a particular embodiment, a single administration is preferred.

According to still further embodiments, the pharmaceutical composition is formulated in a liquid form or in a dry form (e.g., made by freeze drying or spray drying) that is reconstituted in the appropriate solution, buffer or water for injection prior to administration. According to a particular embodiment, the pharmaceutical composition is formulated in a liquid ready for injection. Advantageously, the fibrinogen composition suitable for use in the methods of the present invention is storage-stable between 2° C. to 30° C., preferably it is storage-stable at ambient storage temperatures.

According to another aspect, the present invention provides a method for treating excessive bleeding in a subject suffering from thrombocytopenia comprising administering to the subject in a hospital setting an anti-hemorrhagic pharmaceutical composition consisting of human fibrinogen as the active ingredient, wherein human fibrinogen is selected from the group consisting of fibrinogen purified from human plasma, and recombinant human fibrinogen. According to a certain embodiment, human fibrinogen is human fibrinogen concentrate which is virus free and/or has undergone a plurality of viral inactivation steps and is devoid of blood components.

According to some embodiments, the pharmaceutical composition administered for treating bleeding in the subject suffering from thrombocytopenia is by intravenous injection or infusion. The volume of the pharmaceutical composition for injection is up to 100 ml, alternatively up to 50 ml, further alternatively up to 30 ml, yet further alternatively up to 10 ml. According to a certain embodiment, the pharmaceutical composition is administered by intravenous bolus injection in a volume of up to 50 ml.

According to further embodiments, human fibrinogen administered to the thrombocytopenic subject is present within the pharmaceutical composition in an amount ranging from about 1 g to about 15 g, alternatively from about 2 g to about 10 g, further alternatively from about 2 g to about 5 g, yet further alternatively at about 4 g. The dosage of human fibrinogen to be administered will be determined by the severity of bleeding, the weight and clinical situation of the individual, and the like. Human fibrinogen can be administered in a single dose or multiple times.

According to still further embodiments, the pharmaceutical composition is formulated in a liquid form or in a dry form (e.g., made by freeze drying or spray drying) that is reconstituted in the appropriate solution, buffer or water for injection prior to administration. According to a certain embodiment, the pharmaceutical composition is formulated in a liquid ready for use.

According to yet further aspect, the present invention provides a method for treating or preventing bleeding in a subject suffering from a qualitative platelet disorder comprising administering to the subject in a hospital or pre-hospital setting an anti-hemorrhagic pharmaceutical composition consisting of human fibrinogen as the active ingredient, wherein human fibrinogen is selected from the group consisting of fibrinogen purified from human plasma, and a recombinant human fibrinogen. According to a particular embodiment, the qualitative platelet disorder is Glanzmann's Thrombasthenia. According to another embodiment, the qualitative platelet disorder is Bernard-Soulier disease. According to a certain embodiment, human fibrinogen is human fibrinogen concentrate which is virus free and/or has undergone a plurality of viral inactivation steps and is devoid of blood components. It is to be appreciated that according to the principles of the present invention, human fibrinogen can be used prophylactically to patients suffering from qualitative platelet disorders, and therefore can be administered in pre-hospital settings. Clinicians and/or the individuals can administer the pharmaceutical composition.

According to some embodiments, the pharmaceutical composition administered for treating bleeding in the subject suffering from a qualitative platelet disorder is by intravenous injection or infusion. The volume of the pharmaceutical composition for injection is up to 100 ml, alternatively up to 50 ml, further alternatively up to 30 ml, yet further alternatively up to 10 ml. According to a certain embodiment, the pharmaceutical composition is administered by intravenous bolus injection in a volume of up to 50 ml.

According to further embodiments, human fibrinogen administered to the subject suffering from a qualitative platelet disorder is present within the pharmaceutical composition in an amount ranging from about 1 g to about 15 g, alternatively from about 2 g to about 10 g, further alternatively from about 2 g to about 5 g, yet further alternatively at about 4 g. The dosage of human fibrinogen to be administered will be determined by the severity of bleeding, the weight and clinical situation of the individual, and the like. Human fibrinogen can be administered in a single or multiple times.

According to still further embodiments, the pharmaceutical composition is formulated in a liquid form or in a dry form (e.g., made by freeze drying or spray drying) that is reconstituted in the appropriate solution, buffer or water for injection prior to administration.

According to still further aspect, the present invention provides a method for treating a subject suffering from excessive bleeding having plasma fibrinogen levels above 1-1.5 g/L comprising administering to the subject an anti-hemorrhagic pharmaceutical composition consisting of human fibrinogen as the active ingredient, wherein human fibrinogen is present within the pharmaceutical composition at a range from about 1 g to about 15 g. According to some embodiments, the excessive bleeding is due to any cause selected from the group consisting of traumatic injury, surgery, post-operative bleeding, clinical procedures, open wounds, and internal bleeding such as in liver injury.

According to some embodiments, human fibrinogen is present within the pharmaceutical composition at a range from about 2 g to about 10 g, alternatively from about 2 g to about 5 g, further alternatively at about 4 g.

According to further aspect, the present invention provides use of human fibrinogen for treating excessive bleeding in a pre-hospital setting, wherein human fibrinogen is selected from the group consisting of fibrinogen isolated from human plasma, and a recombinant human fibrinogen according to the principles of the present invention. According to some embodiments, bleeding is due to traumatic injury, open wounds, and internal bleeding such as in liver injury.

According to still further aspect, the present invention provides use of human fibrinogen for treating excessive bleeding in thrombocytopenia, wherein human fibrinogen is selected from the group consisting of fibrinogen purified from human plasma, and a recombinant human fibrinogen according to the principles of the present invention.

According to yet further aspect, the present invention provides use of human fibrinogen for treating excessive bleeding in a qualitative platelet disorder, wherein human fibrinogen is selected from the group consisting of fibrinogen purified from human plasma, and a recombinant human fibrinogen according to the principles of the present invention. According to some embodiments, the qualitative platelet disorder is selected from the group consisting of Glanzmann's Thrombasthenia and Bernard-Soulier disease.

According to further aspect, the present invention provides use of human fibrinogen for treating excessive bleeding when plasma fibrinogen level is above 1-1.5 g/L, wherein human fibrinogen is selected from the group consisting of fibrinogen purified from human plasma, and a recombinant human fibrinogen according to the principles of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. Thrombelastometry (ROTEM) analysis of clotting time at baseline, before administration of fibrinogen concentrate and at the end of the observation period. Data is given in box-plots analysis.

FIG. 2. Thrombelastometry (ROTEM) analysis of clot formation time at baseline, before administration of fibrinogen concentrate and at the end of the observation period. Data is given in box-plots analysis.

FIG. 3. Thrombelastometry (ROTEM) analysis of maximum clot firmness at baseline, before administration of fibrinogen concentrate and at the end of the observation period. Data is given in box-plots analysis.

FIG. 4. Thrombelastometry (FibTEM) analysis of maximum clot firmness at baseline, before administration of fibrinogen concentrate and at the end of the observation period. Data is given in box-plots analysis.

FIG. 5. Thrombelastographic illustration showing the dynamics of development of the clot (CT, CFT and alpha angle) and the clot firmness (MCF).

FIGS. 6A-6C. Thrombelastometry (ROTEM) analysis of clot formation (FIG. 6A), maximum clot thinness (FIG. 6B) and α angle (FIG. 6C) at baseline (1), after platelet apheresis (2), after therapy (3), at the endpoint of observation (4) in the animals treated with platelet concentrate, fibrinogen concentrate or saline. * P<0.05 fibrinogen group vs. platelet group, # P<0.05 fibrinogen group vs. saline group. Δ P<0.05 platelet group vs. saline group for comparison of calculated differences between measurement points 2 and 3 and 3 and 4.

FIG. 7. Rate of blood loss (mL/min) after liver injury in animals treated with fibrinogen, platelets or normal saline. Blood loss velocity occurring after liver injury was significantly greater in the placebo group and in the animals treated with platelet concentrate than in the animals treated with fibrinogen concentrate. * P<0.05 fibrinogen group vs. platelet group, # P<0.05 fibrinogen group vs. the saline group. Δ P<0.05 platelet group vs. saline group.

FIG. 8. Kaplan-Meier analysis: Survival time (min) after liver injury in animals treated with platelets, fibrinogen or normal saline. The survival time following liver injury was significantly longer in the fibrinogen-treated animals than in animals treated with platelets or with saline. * P<0.05 fibrinogen group vs. platelet group, # P<0.05 fibrinogen group vs. saline group. P<0.05 platelet group vs. saline group.

### DETAILED DESCRIPTION OF THE INVENTION

### Definitions

As used herein and in the claims the term “platelet disorders” is intended to include disorders of platelet function or number.

As used herein the term “improved clot formation” refers to either decreased clotting time or increased clot firmness or both.

As used herein, a “recombinant” protein includes those proteins made by recombinant techniques. These proteins include those which resemble the natural protein as well as those modified to enhance activity, protein half-life, protein stability, protein localization and protein efficacy.

An “individual” or “subject” is a vertebrate, preferably a mammal, more preferably a human. Mammals also include, but are not limited to, farm animals, sport animals and pets.

An “effective amount” is an amount sufficient to offer beneficial or desired clinical results. An effective amount can be described in individual amounts, such as the quantity injected (e.g. 3 g fibrinogen material). An effective amount can be administered in one or more administrations. For purposes of this invention, an effective amount of fibrinogen is an amount that is sufficient to cause hemostasis, improve clot formation, decrease bleeding, improve blood coagulation, or decrease blood loss.

As used herein, “hemostasis” is the arrest of bleeding, involving the physiological process of blood coagulation at ruptured or punctured blood vessels and possibly the contraction of damaged blood vessels.

As used herein, “treatment” is a method for obtaining beneficial or desired clinical results. For purposes of this invention, beneficial or desired clinical results include, but are not limited to, alleviation of symptoms, diminishment or preventing of bleeding, stabilization of the individual, preventing bleeding. “Treatment” can also mean prolonging survival of the individual.

As used herein, “bleeding disorder” is defined as decreased ability to control bleeding due to one of the following: vascular defects, thrombocytopenia, thrombocytopathia, defects in blood coagulation or excessive fibrinolytic activity.

As used herein “trauma” is any tissue insult such as an abrasion, incision, contusion, puncture, compression, etc., such as can arise from traumatic contact of a foreign object with any region of the body.

The term “about” is meant to refer to a value, e.g., amount or concentration, which is 10% below or above the indicated value. Thus, an amount of about 2 g is meant to indicate an amount from 1.8 g to 2.2 g.

According to the present invention fibrinogen treatment provides a significant improvement in the impaired dynamics of clot formation and clot firmness, reduces the rate of blood loss, and improves survival in subjects suffering from massive bleeding due to traumatic injury or platelet disorder. It is now disclosed for the first time that fibrinogen alone can be useful to control bleeding in subjects suffering from traumatic hemorrhage, thrombocytopenia, or a qualitative platelet disorder. The methods of the present invention provide treatment of bleeding patients from various causes comprising administering to said subjects “supernormal” doses or levels of fibrinogen so as to improve clot quality and strength and thereby to enhance hemostasis.

The present invention particularly discloses innovative treatment of bleeding related to trauma or platelet disorders using fibrinogen. It is contemplated that the methods of the present invention are particularly applicable to trauma in pre-hospital settings. However, it is also disclosed that in thrombocytopenia and other platelet disorders fibrinogen will be useful both for treatment and prevention of bleeding such as before invasive procedures or even for long term prophylactic treatment in patients with very severe thrombocytopenia who are at high risk to develop dangerous bleedings. It will be understood by persons skilled in the art that certain types of bleeding disorders are explicitly excluded, such as for example patients suffering from thrombotic thrombocytopenic purpura (TTP), a disease of unknown origin characterized by abnormally low levels of platelets in the blood, formation of blood clots in the arterioles and capillaries of many organs, and neurological damage.

Transfusion of platelet concentrate is the traditional treatment for severely thrombocytopenic patients who undergo invasive procedures or suffer bleeding. The exceptions are patients suffering from TTP, HIT or these who are refractory to platelets due to various antibodies. The recommended platelet pretransfusion trigger of 10×10⁹/L for stable non-bleeding hemato-oncological patients has proven to be safe. Even 5×10⁹/L have been suggested to be satisfactory in some studies. For planned invasive procedures (liver biopsy, lumbar puncture, catheter placement, etc.) or surgery, regardless of the type, platelet transfusion is recommended to achieve a platelet count >50×10⁹/L. For neurosurgery and opthalmological surgery involving the posterior segment of the eye a platelet count of 100×10⁹/L is advised, whereas a count of 80×10⁹/L has been proposed for epidural anesthesia. During massive transfusion platelet transfusion is recommended if blood loss is more than two blood volumes. However, all these recommendations for prophylactic transfusion are the result of professional consensus or expert opinion. Studies of the role of platelet transfusion in reducing further blood loss and transfusion requirements are lacking.

It is now disclosed that in patients suffering from massive bleeding due to abdominal trauma or surgical trauma administration of high dose fibrinogen concentrate increased clot firmness and eventually stopped the bleeding.

It is further disclosed that in thrombocytopenic animals administration of high dose fibrinogen concentrate resulted in significantly improved dynamics of clot propagation (CFT, α angle) and final clot strength (MCF) and these effects were stronger as compared to the effects seen after administration of two units of stored apheresis platelet concentrate (equivalent to 12 units of pooled platelets concentrate). The values measured for thrombelastographic variables depend on the interplay of activated platelets with fibrinogen/fibrin and Factor XIII. It seems that in whole blood analysis, reflecting the in vivo situation, a reduction in one of these interacting parameters, namely a decrease in platelets as exemplified herein, can be offset by an increase in others, like fibrinogen/fibrin polymerization, which results in elevated fibrinogen levels following administration of fibrinogen concentrate. It is well known that swine have higher fibrinogen plasma levels than humans do.

Only recently Weinstock et al, on behalf of the SSC Fibrinogen Subcommittee of the ISTH (International Society on Haemostasis and Thrombosis) addressed the analytical problems of plasma samples containing large amounts of fibrinogen and suggested a new fibrinogen 500 mg dL⁻¹ standard (Weinstock et al., J. Thromb. Haemost. 4: 1825-1827, 2006). In light of such analytical problems it is not surprising that the plasma fibrinogen levels measured before and after administration of fibrinogen concentrate using standard methods showed only minor (although statistically significant) differences, while the corresponding functional ROTEM® measurements (alpha angle, CFT, MCF) changed more dramatically.

It is also possible to use the ELISA technique to analyze study samples for content of human fibrinogen. The ELISA results strongly support the hypothesis that ROTEM® results reflected true fibrinogen plasma levels as opposed to those obtained with the Claus method.

However, an improvement in MCF following administration of fibrinogen has also been seen during dilutional coagulopathy, namely decreased fibrinogen concentrations but platelet numbers within safe limits (Fries et al., Br. J. Anaesth 95: 172-177, 2005). These results were also confirmed ex vivo by Fenger-Erikson et al. (Fenger-Erickson et al., Br. J. Anaesth 94: 324-329, 2005). Increased bleeding after cardiopulmonary bypass is significantly associated with decreased fibrinogen levels (Fries et al., Anesth. Analg. 99: 947, 2004; Blome et al., Thromb. Haemsot. 93: 1101-1107, 2005; Pothula et al., Anesth. Analg. 98: 4-10, 2004). Interestingly, high fibrinogen values have been shown to protect against blood loss during major surgical procedures.

Integrity of the hemostatic system is also essential for the safety of neurosurgical procedures. Patients with decreased factor XIII showed an increased risk for developing postoperative hematoma requiring surgical evacuation. Notably, this risk increased dramatically in cases also showing moderately reduced fibrinogen levels and platelet numbers.

One concern associated with the administration of fibrinogen concentrate is the development of thrombosis and thrombembolic complications. High plasma fibrinogen levels are associated with an increased risk for coronary heart disease and stroke and are associated with higher plasma viscosity and increased risk for cardiovascular events including ischemic heart disease and stroke. The efficacy and tolerability of pasteurized human fibrinogen concentrate (Haemocomplettan P®, ZLB Behring, Marburg, Germany) were retrospectively studied in patients with only congenital fibrinogen deficiency (Kreuz et al., Transfus. Apher. Sci. 32: 247-253, 2005); one of these patients developed a deep vein thrombosis and a non-fatal pulmonary embolism after hip fracture. However, it is known that patients with hypo- or dysfibrinogenemia are prone to thrombembolic complications after fibrinogen concentrate or cryoprecipitate administration (Kreuz et al., ibid). Further reports on thrombembolic events due to the use of fibrinogen concentrate are lacking in the literature. However, there are also reports on thrombembolic complications following the administration of fresh frozen plasma.

It is now disclosed that histological examination after fibrinogen administration did not detect microvascular thrombosis in the lungs, heart, gut, spleen or liver in animals in which thrombocytopenia and uncontrolled hemorrhaging were induced. D-dimer as a laboratory parameter of this phenomenon was elevated only at the end of the observation period in the animals treated with fibrinogen, while TAT did not differ between groups. In addition, D-dimer values between 200 and 300 μg/L can be interpreted as an adequate response to liver injury. Furthermore, thrombelastographic parameters after fibrinogen administration did not show any signs of hypercoagulability, since all parameters after therapy with fibrinogen concentrate were in the range of baseline measurements.

Transmission of infection is a further side effect of administration of plasma products. Clotting factor concentrates isolated from plasma undergo several virus inactivation steps and can be considered much safer compared to non-virally inactivated blood products such as fresh frozen plasma, platelet concentrate or cryoprecipitate. In addition, pasteurized fibrinogen concentrate is free of contaminating leukocytes and thus extremely unlikely to provoke transfusion-related lung injury (TRALI).

Certain limitations to the specific animal models used to exemplify the present invention need to be noted. For the sake of comparability and standardization, apheresis and fibrinogen or platelet administration had to be performed immediately before liver incision. The study in an animal model of thrombocytopenia was not designed to simulate liver trauma or its potential treatment. Liver injury was inflicted to study the clinical consequences of bleeding due to trauma or thrombocytopenia and following administration of fibrinogen concentrate or platelets and to compare these consequences with surrogate markers of bleeding tendency including ROTEM® results.

Moreover, low platelet count is often not associated with massive bleeding in nonsurgical patients. If invasive procedures or surgery are indicated, however, prophylactically transfusion of platelet concentrates is generally recommended. From this point of view, the model of the present invention mimics the clinical situation quite well.

The effect of fibrinogen concentrate on bleeding due to platelet disorders including thrombocytopenia has never been investigated before, neither in an experimental model or in vivo. As demonstrated herein below in thrombocytopenic pigs, administration of fibrinogen improved hemostasis, decreased the rate of blood loss and prolonged survival time after liver injury better than did the commonly practiced transfusion of platelet concentrates. Thus, the administration of fibrinogen concentrate may be a useful first step in reducing the need for platelet concentrate when bleeding takes place.

### Human Fibrinogen

The human fibrinogen protein ordinarily circulates in high quantities in plasma (2-4 g/L). Fibrinogen acts as a plug substrate for sealing vessel injury sites. At times of injury, the body is stimulated to produce excess amounts of fibrinogen. The activation response to produce increased quantities of fibrinogen produces levels of fibrinogen 2-3 times the normal level. This upregulation and production of fibrinogen takes approximately 1-2 days, at which time large quantities of blood loss may have already occurred. This delayed process is often ineffectively late in cases of severe bleeding or bleeding at critical sites. Introduction of fibrinogen will increase the concentration of fibrinogen in the plasma in a shorter time period. Thus, the introduction of excess fibrinogen will allow the extrinsic coagulation pathway to occur without being hindered by a lack of fibrinogen. Plasma and blood products have been used to replenish the diminished supply of fibrinogen in the past. A single dose of 3-10 grams of fibrinogen is equivalent to the fibrinogen content of 10-25 units of fresh frozen plasma or cryoprecipitate.

Fibrinogen is further defined as any whole fibrinogen polypeptide or functional equivalent including, but not limited to, deletions, insertions, mutations, modifications, truncations and transpositions of amino acids from the polypeptide sequence. The functionality of fibrinogen or a functional equivalent can be tested by performing a prothrombin assay to determine the effectiveness of the polypeptide on blood coagulation time.

It is now possible to manufacture injectable fibrinogen solutions which are essentially virus-free. Fibrinogen may be produced by bulk purification from plasma and followed by further purification with detergent treatment or other means to inactivate contaminants such as viral contaminants, for example Hepatitis virus particles. Advantageously a plurality of virus inactivation methods may be employed. The fibrinogen isolated from human plasma is devoid of blood components, e.g., factor VIIa. Alternatively, fibrinogen is commercially available from multiple companies (e.g., Aventis Behring, Baxter, Alpha Therapeutics). Alternatively, fibrinogen can be obtained from recombinant sources, such as are known in the art. Recombinant fibrinogen can be transgenically produced, for example, from plants or in the milk of sheep or cows. Recombinant human fibrinogen can also be obtained in expression systems using host cells including, but not limited to, CHO, BHK, COS cells, or other eukaryotic host cells, prokaryotic host cells, yeasts or other fungi, according to procedures as are known in the art.

A recombinant form of fibrinogen polypeptide will retain the functional characteristics of native fibrinogen polypeptide. The benefit of using the recombinant form of fibrinogen is that large quantities can be safely, effectively and economically produced, while minimizing the risk of contaminants, particularly of viral contaminants.

The fibrinogen polypeptide may be modified in a number of ways including, but not limited to, chemical modification, glycosylation, methylation, hydroxylation, amino acid deletion, insertion, mutation, truncation and transposition, as long as the polypeptide retains the haemostatic activity.

### Pharmaceutical Compositions

The pharmaceutical composition of the present invention consists essentially of human fibrinogen and is used to cause hemostasis, blood clotting, decrease of blood loss and/or affect blood coagulation. The pharmaceutical composition of the present invention can be manufactured and kept stable in a variety of dry and wet forms. The dry powders, e.g., freeze dried or lyophilized, or the liquid solutions can be mixed, sterilely packaged, and stored for years dry or months wet (see, for example, U.S. Pat. No. 7,045,601, the content of which is incorporated by reference as if fully set forth herein). Stable fibrinogen is particularly useful in pre-hospital settings where the fibrinogen composition can be stored at ambient temperatures, such as in combat fields, natural disasters, in ambulances, or in any other pre-hospital emergency. When fibrinogen is isolated from human plasma by chromatography, it is present in essentially pure form, and therefore the use of this pharmaceutical composition is safer than administering fresh frozen plasma, since it eliminates complications associated with other blood products (e.g. blood typing, foreign matter, viral contaminants).

The pharmaceutical composition may further comprise other ingredients added to improve the stability of fibrinogen, such as ions, e.g., monovalent or divalent metal ions, sugars, polysaccharides, such as low molecular weight dextrins, polyalcohols such as glycerol, antioxidants, such as bisulfite or ascorbate, albumin, complexing agents, and buffers. The stabilizing agents that stabilize the protein during the lyophilization process and/or storage are generally present in a concentration from 0.1 to 5% weight/volume. The dried or lyophilized composition can be rehydrated easily in solution with appropriate excipients, such as, but not limited to, sucrose solution, saline distilled water, or any pharmaceutically acceptable carrier.

Fibrinogen is relatively less soluble than some other plasma proteins. 10% solutions (1 g/10 mL) are feasible, whereas solutions above 15% are viscous and difficult to rehydrate. Attempts at modifying fibrinogen have resulted in decreased solubility. A normal individual weighing 70 kg has about 3 liters of plasma volume each containing 2-4 g/L of fibrinogen. A dose of 3 grams, which would insure the minimum plasma fibrinogen concentration of 1 g/L and raising the plasma concentration by 25-50% can be formulated and administered in as little as 30 mL. Fibrinogen can be administered in a dosage effective to produce in the plasma an effective level of about up to 12.0 g/L, preferably 0.25-10.0 g/L, more preferably 0.5-6.0 g/L, although fibrinogen may be administered in higher quantities. Factors that may be involved in determining the amount of fibrinogen administered include the amount of fibrinogen suspected to be lost through bleeding, the number and severity of hemorrhaging sites, the location of injection(s), and the general physical condition of the individual. For example, higher overall fibrinogen quantities may be achieved by multiple injections of 6.0-12.0 g injections near multiple sites of hemorrhaging injury.

An individual who has a bleeding episode can be re-assessed and re-administered the composition if bleeding has not decreased to an acceptable level. An acceptable level is defined as bleeding that is controlled and does not pose any threat to the life of the individual or cause any detrimental harm to the individual. The composition may be administered at time intervals of about 5-10 hours, or may be administered at time intervals of as little as 0.5-2 hours. It must be noted that fibrinogen has a limited biological half-life, which may affect the frequency of administration. Thus, it may be beneficial to administer smaller doses more frequently. The composition of the current invention may be administered by bolus injection or by continuous infusion; the method of administration should be reflective of the purpose of administration. For example, if there is severe bleeding and complete or partial coagulation or decrease in bleeding is desired, a bolus injection would be preferred. In cases of prophylactic use, such as during controlled minor surgical procedures, a method of continuous infusion may be used.

The following examples are provided to illustrate but not limit the invention.

### EXAMPLES

### Example 1

### External Bleeding in Pre-Hospital Settings

An individual suffers from multiple external injuries as a result of a car accident, terrorist attack or any other trauma. As the individual cannot be immediately transported to a hospital the physician or paramedic attempts to minimize bleeding in order to maximize the chances of bringing the individual to a hospital alive. Initial treatment includes tourniquets and haemostatic bandages to slow the bleeding. The individual does not experience sufficient decrease in bleeding and fibrinogen is prepared for injection. Three grams of lyophilized fibrinogen are dissolved in 30 ml of saline and shaken until fibrinogen is fully dissolved. The solution is injected intravenously so that fibrinogen can circulate to sites of injury. The individual is reassessed for bleeding following 30 minutes observation, and if bleeding has not decreased to a controllable level, then a second injectable fibrinogen solution is administered. Alternatively, a liquid fibrinogen composition ready for use is administered.

Alternatively, an individual suffering from multiple external injuries is treated by application of tourniquets and haemostatic bandages to slow the bleeding. The individual receives 250 ml of hypertonic saline and the hemodynamic parameters, e.g. blood pressure, are measured. If the blood pressure decreases, fibrinogen is prepared for injection. Three grams of lyophilized fibrinogen are dissolved in 30 ml of saline and shaken until fibrinogen is fully dissolved. The solution is injected intravenously so that fibrinogen can circulate to sites of injury.

### Example 2

### Internal Bleeding in Pre-Hospital Settings

When bleeding is internal in a multiple traumatized patient, the decision to administer fibrinogen concentrate has to be based on surrogate parameters. While heart rate often fails to determine the presence of major bleeding, hemoglobin measurement and blood gas analysis (determination of base excess) help to detect a clinical relevant bleeding in a patient with internal bleeding.

Hemoglobin measurement is performed with the Haemocue analyzer (HemoCue GmbH, Grossostheim, Germany) to detect relevant blood loss. Hemoglobin levels (Hgb) below 10 g/dL indicate the presence or absence of bleeding. Thus, a Hgb value below 10 g/dL is a good indication of internal bleeding patients to whom early fibrinogen administration is beneficial.

Negative base excess measured with a blood gas analyzer provides evidence of a hypovolemic/hemorrhagic shock which implies that significant blood loss occurs in a multiple traumatized patient.

Both methods of hemoglobin measurement and blood gas analysis can be performed using suitable analyzers in the field.

Patients with internal bleeding receive a dosage of 50 mg/kg bodyweight immediately. A contraindication is in patients with high risk for thromboembolism.

### Example 3

### Case Report: Administration of Fibrinogen Concentrate Following Abdominal Trauma and Splenic Rupture

A 12 year old boy fell from his scooter. The abdominal sonography showed a traumatic rupture of the spleen as well as a huge amount of blood in the abdominal cavity. At that time, estimated blood loss was about 700 mL (25% of the estimated total blood volume). After stabilization of blood pressure with crystalloids and colloids, clot firmness as well as all other standard coagulation tests decreased significantly. After administration of 3 g fibrinogen concentrate (Haemocomplettan, CSL, Marburg, Germany) coagulation improved again. Bleeding stopped after laparatomy without the need for splenectomy or transfusion of any allogeneic red blood cell concentrates. The boy recovered completely without any further bleeding or thromboembolic complications.

### Example 4

### Case Report: Administration of Fibrinogen Concentrate Following Surgical Trauma

Ten patients underwent major orthopedic procedures with a blood loss ranging from 30% to 180% of the estimated total blood volume. All patients had a clinically relevant microvascular bleeding tendency as well as a decreased amplitude in the thrombelastographic monitoring. After administration of 2 g-4 g fibrinogen concentrate, microvascular bleeding tendency decreased and clot firmness increased.

### Data Collection

Ten patients, 4 men and 6 women at a mean age of 27 years (12-64 years) underwent surgical correction of idiopathic scoliosis. The patients were anesthetized with fentanyl, propofol and rocuronium by maintenance of anesthesia with inhalated sevofluorane and bolus injection of fentanyl. Preoperative monitoring included radial and central vein catheters in addition to routine noninvasive monitoring, including peripheral nerve stimulator.

Blood samples were collected preoperatively as well as immediately before and after administration of fibrinogen concentrate (Hemocompletan®, CSL, Marburg, Germany). The coagulation analysis included thrombelastographic monitoring (ROTEM® Pentapharm, Munich, Germany) and routine laboratory methods using prothrombin time (PT, normal range 70%-120%), activated partial thromboplastin time (aPTT, normal range 23-40 s), Clauss derived fibrinogen concentration (Fib, normal range 190-380 mg/dL), Antithrombin (AT, normal range 80-120%), platelet count and hemoglobin.

Surgical blood loss was compensated with Ringer's solution (RL) (Fresenius, Pharma Austria Co., Graz, Austria), 4% gelatin (Gelofusin®, B. Braun Co., Melsungen, Germany), red blood cell concentrates and cell saver concentrate to maintain central venous and arterial pressure at about 20% below baseline values.

In addition to the standard laboratory analysis, coagulation status during surgery was monitored with the POC suitable ROTEM® system. In cases where the maximum clot firmness in the extrinsic activated thrombelastographic measurement (ExTEM®, Nobis Co., Endingen, Germany) decreased in combination with the fibrin polymerization amplitude (FibTEM®, Nobis Co., Endingen, Germany) below 10 mm in association with an increased microvascular bleeding tendency, fibrinogen concentrate was administered. The standard laboratory coagulation analysis had no influence on the decision of the administration of fibrinogen concentrate, because of the delay of the test results of more than 40 minutes.

All patients recovered from anesthesia and were discharged from hospital without apparent adverse sequelae. There were no congenital coagulation disorders observed in these patients. Further, none of the patients received any anticoagulant or antiplatelet medication during the last two weeks during or before the surgical procedures.

### Results

The preoperative coagulation values (PT, aPTT, Fib and platelets) as well as the ROTEM® measurements were all in the normal range. Until the need of fibrinogen concentrate substitution reached, estimated median blood loss was about 2,200 mL (550-3,000 mL). At that time, the patients received 2,500 mL of RL (2,000-4,500 mL), 1,650 mL gelatin solution (500-2,500 mL), two units of red blood cell concentrate (0-4 units) and 530 mL of cell saver concentrate (150-920). Within the further observation period, surgical blood loss continued. At the end of the observation period, the patients received 4,000 mL of RL (2,300-5,000 mL) and 2,750 mL of gelatin solution (1,500-4,500 mL). None of the patients received fresh frozen plasma or platelet concentrates, while the estimated median blood loss was 3,250 mL (1,100-4,500) at this time.

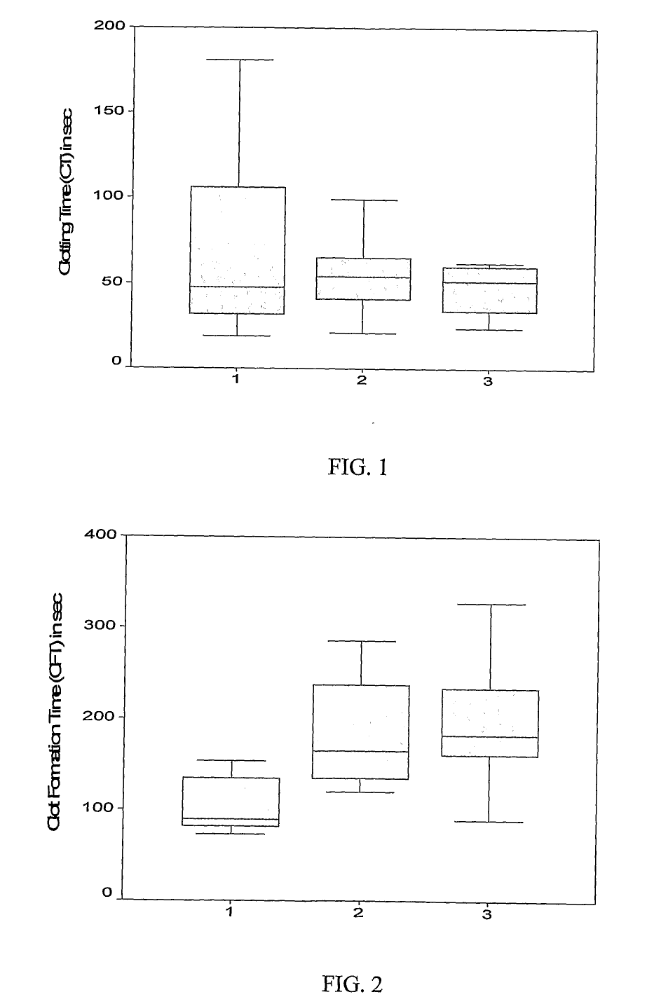

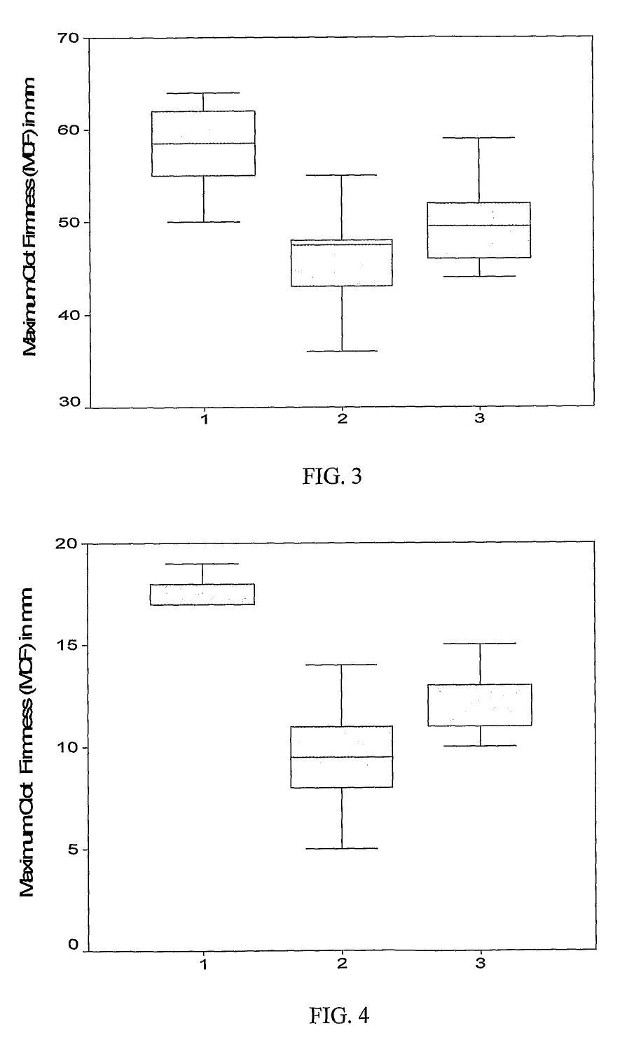

During the observation period, fibrinogen and PT decreased, while aPTT increased. Platelets decreased as well but never reached critical values. From the clinical aspect, all patients had an increased microvascular bleeding tendency, which was associated with impaired amplitudes in the ROTEM® analysis compared to the baseline measurements, showing a clinically relevant decreased clot firmness in the ExTEM® and FibTEM® measurements. The patients received 2-4 g fibrinogen concentrate, depending on body weight, results of the ROTEM®-measurements and on the clinically bleeding tendency. In spite of ongoing surgical blood loss, microvascular bleeding tendency decreased and clot firmness increased (FIGS. 1 to 4).

### Example 5

### Case Report: Use of Fibrinogen in a Patient Unresponsive to Hemostatic Interventions

A 27-year-old woman was admitted to the intensive care unit (ICU) because of septic shock one day after a Cesarean section. Initially, she presented with symptoms of an acute abdomen. Intraoperative examination showed severe cellulitis and necrotizing fasciitis of the abdominal region and diffuse peritonitis. Fasciotomy and debridement were performed. On arrival at the ICU, she became anuric, hemodynamically unstable and needed maximum catecholamine supply with epinephrine, norepinephrine and vasopressin.

The patient developed massive abdominal wall bleeding associated with severe disseminated intravascular coagulation (DIC) after the first surgical intervention. Coagulation analysis revealed a platelet count of 21 G/L, prothrombin time (PT) of 22%, an activated prothrombin time (aPTT) of longer than 78 sec, plasma fibrinogen levels of 170 mg/dL, antithrombin (AT) at 22% and D-Dimer (DD) at 2,939 μg/L. Serum biochemistry showed lactate at 250 mg/dL combined with severe metabolic acidosis. Transfusion of about four red blood cell concentrates (RBC) every hour became necessary for the next 22 hours. Several surgical interventions failed to stop bleeding from the abdominal wall. Coagulation therapy included administration of platelet apharesis concentrates (PLT), desmopressin (Octostim, Ferring, Vienna, Austria), fresh-frozen plasma (FFP), 1 million IU aprotinin (Pantinol, Gerot Parmazeutika, Vienna, Austria), and prothrombin complex concentrate (Beriplex®, Aventis Behring, Marburg, Germany), while platelet transfusion failed to increase platelet count significantly due to increased consumption as a result of severe DIC.

Despite normal plasma fibrinogen values, fibrinogen concentrate was administered (Hemocompletan®, Aventis Behring, Marburg, Germany) to increase maximum clot firmness, which was decreased due to thrombocytopenia (platelet count of 21 G/L). Therapy was guided by modified thrombelastography (ROTEM, Pentapharm Munich, Germany).

Subsequently, recombinant activated factor VII (rFVIIa) was administered in three doses of 100 mg/kg each. After normalization of coagulation in combination with local application of fibrin glue and tranexamic acid, bleeding stopped. The patient was finally discharged alive from hospital after four months.

This case report summarizes the course of treatment of a severely thrombocytopenic patient unresponsive to platelet transfusion, where administration of fibrinogen concentrate was employed successfully to increase clot firmness.

### Example 6

### Use of Fibrinogen in Animal Models of Thrombocytopenia

### Materials and Methods

The study was approved by the Austrian Federal Animal Investigation Committee, and the animals were managed in accordance with the American Physiological Society institutional guidelines, and the Position of the American Heart Association on Research Animal Use, as adopted on Nov. 11, 1984. Animal care and use were performed by qualified individuals supervised by veterinarians, and all the facilities and transportation comply with current legal requirements and guidelines. Anesthesia was used in all surgical interventions, all unnecessary suffering was avoided, and research was terminated if unnecessary pain or distress resulted. Animal facilities meet the standards of the American Association for Accreditation of Laboratory Animal Care.

### Surgical Preparations and Measurements

This study was performed in 30 healthy, 12- to 16-week-old swine weighing 40-45 kg.

The animals were fasted over night, but had free access to water. The pigs were pre-medicated with azaperone (4 mg kg⁻¹ i.m., Stresnil™, Janssen, Vienna, Austria) and atropine (0.1 mg kg⁻¹ i.m.) 1 h before study commencement. Anesthesia was induced with ketamine (20 mg kg⁻¹ i.m.) and propofol (1-2 mg kg⁻¹ i.v.) and maintained with propofol (6-8 mg kg⁻¹ h⁻¹ i.v.). Analgesia was performed with piritramid (30-45 mg i.v., Dipidolor®, Janssen, Vienna, Austria). Pancuronium (0.2 mg kg⁻¹ i.v.) was administered after intubation as a muscle relaxant in order to facilitate laparotomy. After intubation the pigs were ventilated with oxygen 35% using a volume-controlled ventilator (Draeger EV-a; Lubeck; Germany) at a rate of 20 breaths per minute and a tidal volume adjusted to maintain normocapnia. After inducing narcosis the femoral artery and jugular vein were dissected. A 6 Fr catheter was advanced into the femoral artery for collection of blood samples and continuous arterial pressure monitoring. A 12 Fr large bore catheter was advanced into the right jugular vein for apheresis and central venous pressure monitoring. The baseline fluid requirement (4 mL kg⁻¹ h⁻¹ i.v.) was substituted with crystalloid (Ringer's lactate) via a peripheral venous access during the entire course of the procedure. Body temperature was maintained between 38.0° and 39.0° C.

### Experiment Protocol

After induction of anesthesia and insertion of catheters a midline laparotomy and splenectomy were performed; 15 minutes after splenectomy baseline coagulation parameters were obtained (measurement point 1). Platelets were depleted by apheresis using an Amicus® cell separator (Baxter Health Corporation, Deerfield, Ill., USA). Under aseptic conditions the apheresis system was connected to the internal jugular vein with a 12 Fr large bore catheter.

Draw and return blood flow was limited to 150 mL per minute as described in detail in the producer's manual. Platelets were discontinuously collected and resuspended in autologous plasma. From one donor animal two units of apheresis platelet concentrate (one unit of apheresis platelet concentrate corresponds to six units of pooled platelet concentrate) were separated.

A platelet count of less than 30×10⁹/L (measurement point 2) was defined as the endpoint of the apheresis procedure. After a resting period of 1 h the platelets were stored between 20° and 24° C. under continuous shaking. Transfusion was performed on day 3 after apheresis. Thereafter, the animals in group A received two units of homologous apheresis platelet concentrate from one donor animal to achieve a platelet count of more than 50×10⁹/L in the recipient animal in accordance with the recommendations for maintaining blood platelet count in bleeding patients or in those undergoing invasive procedures at >50×10⁹/L. The animals in group B were treated with 250 mg/kg fibrinogen concentrate (Haemocomplettan® P, ZLB Behring, Marburg, Germany). This dose of fibrinogen concentrate has been shown to restore maximum clot firmness (MCF) in coagulopathic pigs in previously published animal experiment data (Fries et al., 2005. ibid).

The animals in Group C (placebo group) were infused with an equal amount of normal saline (NaCl 0.9%). Following substitution all values were measured again (measurement point 3). In order to determine and compare the clinical effect of the above therapies, a hepatic incision (7 cm long and 1.5 cm deep, standardized with a template and always performed by the same blinded examiner) was made in the right liver lobe to induce uncontrolled hemorrhage (central to the falciform ligament above the central lobe). The time to death from hemorrhagic shock was determined and at the end of the study protocol, blood was suctioned out of the abdomen and the total blood loss measured. If an animal died within the first 120 min, the last blood sample was taken immediately before the anticipated death, which was defined as pulseless electrical activity, mean arterial pressure below 10 mm Hg and an end tidal carbon dioxide below 10 mm Hg. Animals surviving more than 2 h were sacrificed with an overdose of piritramid, propofol and potassium chloride (measurement point 4: 120 min after liver incision or immediately before anticipated death). Those investigators who performed coagulation analysis, documentation of hemodynamics, liver incision and collection and measurement of shed blood were blinded to the treatment group. The circulatory situation was not influenced by catecholamines or volume substitution.

### Blood Sampling and Analytical Methods

Arterial blood sample collection was performed at baseline, following apheresis, platelet transfusion or administration of fibrinogen or normal saline, as well as 120 min after liver injury or immediately before anticipated death. All blood samples were drawn from the femoral artery, whereby the first ten milliliters of blood were discarded. Blood samples for modified thrombelastometry (ROTEM®, Pentapharm, Munich, Germany) and standard coagulation analysis were collected in 3-mL tubes containing 0.3 mL (0.106 mol/L) buffered (pH 5.5) sodium citrate (Sarstedt, Nuermbrecht, Germany). Blood samples for blood cell count were collected in 2.7-mL tubes containing 1.6 mg EDTA/mL (Sarstedt, Nuermbrecht, Germany). All tests were performed by the same investigator.

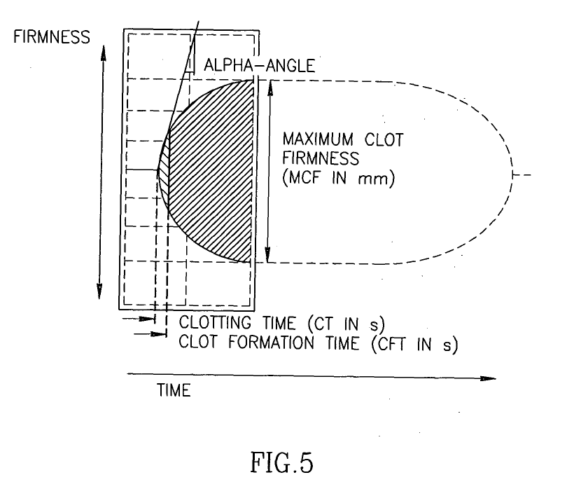

Prothrombin time (PT), partial thromboplastin time (aPTT), concentrations of fibrinogen (Clauss method), antithrombin (AT) and thrombin-antithrombin complexes (TAT) were determined by standard laboratory methods using the appropriate tests from Dade Behring, Marburg, Germany, and the Amelung Coagulometer, Baxter, UK. For determination of D-Dimer the assay D-Dimer-0020008500® (Instrumentation Laboratory Company, Lexington, USA) was used. Human fibrinogen concentration was measured with the paired antibodies Cedarlane® ELISA for fibrinogen antigen (Biozol Company, Eching, Germany). Blood cell count was performed with the Sysmex Poch-100i® counter (Sysmex, Lake Zurich, USA) (e.g. platelets and red and white blood cells). Thrombelastography was performed with the Rotem® coagulation analyzer (Pentapharm, Munich, Germany) using the in-TEM® assay (Pentapharm, Munich, Germany). The following variables were determined: CT (sec, clotting time) corresponding to the reaction time (r) in a conventional thrombelastogram, CFT (sec, clot formation time) corresponding to the coagulation time (k), MCF (mm, maximum clot firmness), which is equivalent to the maximum amplitude (MA), and the α angle (FIG. 5).

Postmortem autopsy of the animals was performed and heart, lungs, liver, spleen and parts of the intestine of the deceased animals were removed and macroscopically and histologically explored for thrombosis.

### Statistical Analysis

A non-parametric Friedmann ANOVA was applied to determine a possible time effect in each group. Calculated differences between the measurement points 2 and 3 as well as between 3 and 4 were compared between the various groups using the Wilcoxon test for unpaired observations. Thrombelastometric parameters and blood loss are presented in box plots (minimum, first quartile, median, third quartile, maximum). A Kaplan-Meier analysis was performed for survival time analysis. A P value less than 5% was considered statistically significant. Data are presented as median and interquartile range (25th to 75th percentile), if not otherwise indicated.

The sample size of ten animals per group allowed for the detection of a difference in the mortality rates between the placebo (90%) and the fibrinogen (30%) group (two-sided alpha=5%, power=80%).

### Results

At baseline, pigs were comparable with regard to hemodynamic and coagulation parameters as well as platelet and red blood cell count (Table 3). All coagulation tests were within the normal reference intervals described for pigs.

After apheresis hemoglobin decreased only marginally, while platelet counts decreased to the targeted critical level of approximately 30×10⁹/L in all three groups. As expected, platelet count increased significantly in the platelet transfusion group only (Table 3). Fibrinogen concentrations decreased slightly in all groups after apheresis. After administration of the study drugs, fibrinogen values increased significantly in the animals treated with fibrinogen concentrate.

Furthermore, a mild increase in fibrinogen was also seen in the animals treated with platelet concentrate (Table 3). As expected, using an ELISA technique, human fibrinogen was detected only in the animals treated with fibrinogen concentrate (Table 3). Neither PT nor aPTT changed due to apheresis or study drug administration, whereas antithrombin decreased slightly in all groups (Table 3).

D-Dimer values increased significantly only at the end of the study in the fibrinogen-treated pigs, while TAT did not differ between the groups over the whole observation period (Table 3).

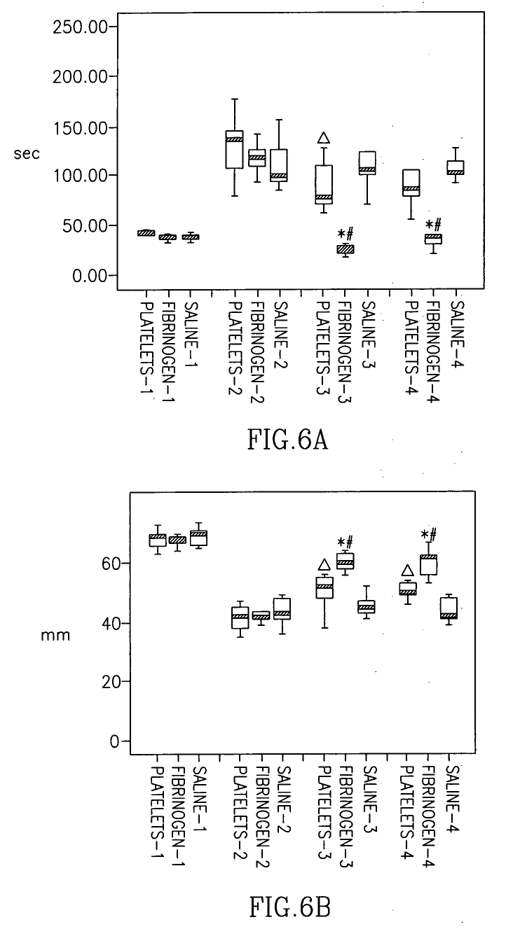

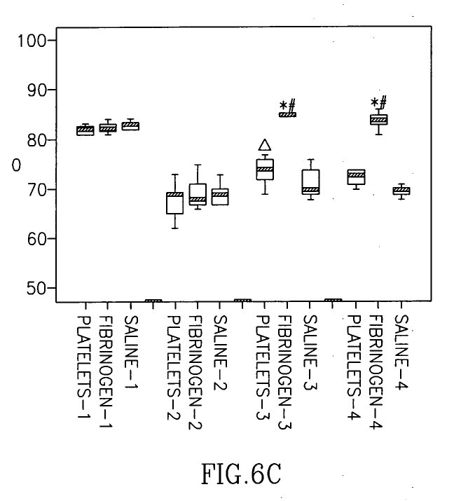

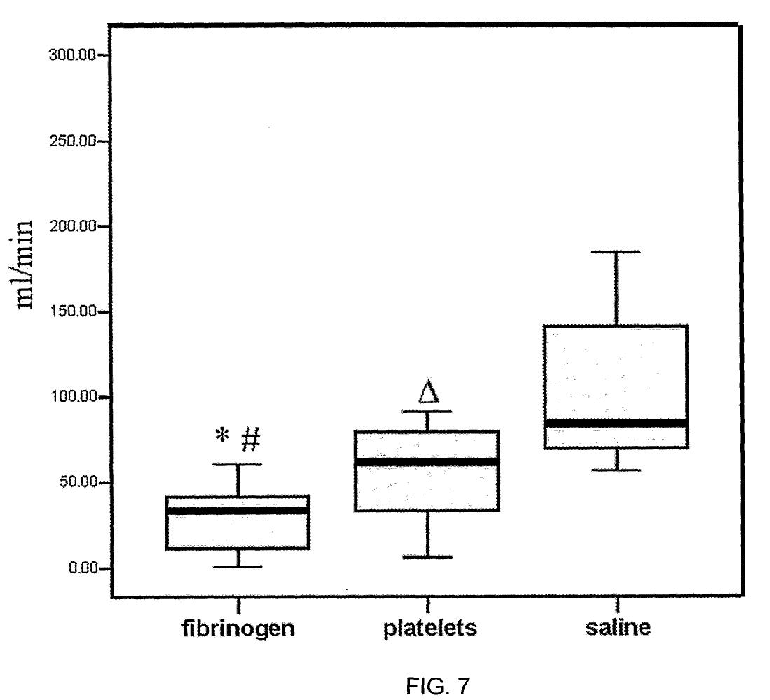

After administration of fibrinogen concentrate CFT shortened significantly more than following platelet transfusion (P=0.0002) or placebo (P=0.0002) (FIG. 6A) and MCF increased significantly more after fibrinogen administration than in animals who received platelet concentrate (P=0.0004) or saline (P=0.0002) (FIG. 6B). Median (Q1, Q3) blood loss velocity occurring after liver injury was significantly greater in the placebo group (84 ml/min; 68, 152), (P=0.005) and in the animals treated with platelet concentrate (62 ml/min; 33, 81), (P=0.037) than in the animals treated with fibrinogen concentrate (33 ml/min; 10, 45). As expected, median blood loss velocity was also significantly less in animals treated with platelet concentrate than in the control group (P=0.017) (FIG. 7).

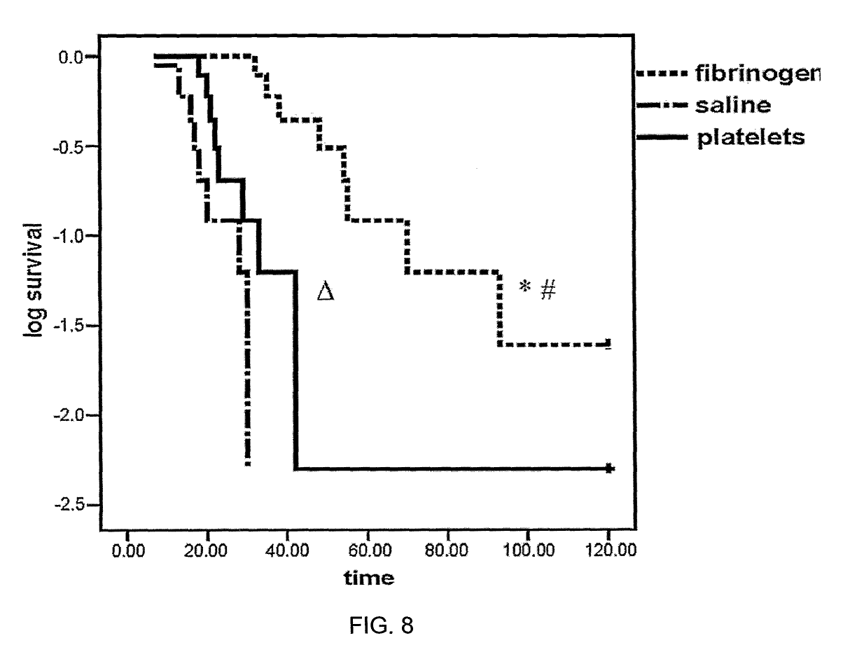

The survival time following liver injury was significantly longer [55 min (37, 100) in the fibrinogen-treated animals than for those treated with platelets [26 min (21, 42)], (P=0.035) or for those treated with saline [19 min (15, 30)], (P=0.0000) (FIG. 8).

Animals treated with platelet concentrates survived significantly longer (P=0.049) than did animals in the placebo group (FIG. 8). Twenty percent of the fibrinogen-treated animals as compared to 10% of the platelet-treated animals (P=0.0072) and none of the saline-treated group survived the 2 h observation period (P=<0.0001).

Autopsy showed thrombus formation in the pulmonary artery of one animal treated with fibrinogen concentrate. Histological examination determined a white thrombus (3×35 mm) assuming that it was generated in an agonal state in the line of ventricular fibrillation. No microvascular thrombosis was detected in the lungs, heart, gut, spleen or liver of the control animals or of the pigs treated with platelet or fibrinogen concentrate.

### Example 7

### Use of Fibrinogen for Treating Glanzmann's Thrombasthenia (GT)

Patients with GT often become refractory to platelets transfusions. Recombinant factor VIIa (rFVIIa) has been approved for such patients but the response is unpredictable and in some cases disappointing. Our previous studies in animals and humans showed that high doses of fibrinogen concentrate are favorable in the prevention and treatment of bleeding in various clinical situations. As shown herein above (Example 6), high dose fibrinogen concentrate reduced blood loss and increased survival in an animal model of traumatized thrombocytopenic pigs (30,000 plt/μL) better than platelets transfusions. These findings raise the possibility that high dose fibrinogen may be effective in GT and possibly in other qualitative platelets defects.

Severe GT patient constantly bleeding from many mucocutaneous sites is treated with 4 g fibrinogen concentrate (Hemocompletan™, Aventis Behring, Germany). ROTEM parameters of clot formation and firmness are improved within one hour post infusion. Bleeding stops for a month.

Thus, high dose fibrinogen concentrate may be effective in the treatment/prevention of bleeding in GT patients.

Although the foregoing invention has been described in some detail by way of illustration and example for purposes of clarity of understanding, it will be apparent to those skilled in the art that certain changes and modifications can be practiced. Therefore, the description and examples should not be construed as limiting the scope of the invention, which is delineated by the appended claims.

TABLE 1
Median (Q1, Q3) of hemoglobin, platelet count, fibrinogen concentration, PT, aPTT,
AT, D-Dimer, TAT, at baseline, after platelet apheresis to approximately 30 × 10⁹/L, after
therapy, and at the end of the observation period (120 min after therapy).

| | | | After platelet | | | | 120 min after | |
|---|---|---|---|---|---|---|---|---|
| | Baseline | | apheresis | | After therapy | | therapy | |
| Hemoglobin (g L⁻¹) | | | | | | | | |
| Platelet concentrate | 97 | (92-104) | 85 | (81-92) | 81 | (72-89) | 62 | (57-73) |
| Fibrinogen | 96 | (85-100) | 80 | (75-90) | 77 | (71-81) | 53 | (37-64)^{#} |
| Saline | 100 | (92-104) | 84 | (82-98) | 84 | (81-92) | 72 | (64-79) |
| Platelet count (10⁹ L⁻¹) | | | | | | | | |
| Platelet concentrate | 316 | (270-348) | 30 | (27-30) | 55 | (52-62)^{Δ} | 51 | (50-63) |
| Fibrinogen | 317 | (247-361) | 31 | (28-33) | 33 | (28-40)* | 35 | (23-39) |
| Saline | 308 | (266-346) | 30 | (28-31) | 31 | (26-35) | 31 | (27-41) |
| Fibrinogen (mg dL⁻¹) | | | | | | | | |
| Platelet concentrate | 357 | (319-410) | 255 | (236-334) | 330 | (274-369)^{Δ} | 250 | (242-317)^{Δ} |
| Fibrinogen | 303 | (258-366) | 235 | (202-257) | 341 | (298-376)*^{#} | 281 | (235-370)^{#} |
| Saline | 346 | (317-402) | 254 | (233-275) | 274 | (231-249) | 233 | (211-250) |
| Human fibrinogen (mg | | | | | | | | |
| mL⁻¹) | | | | | | | | |
| Platelet concentrate | nd | | nd | | nd | | nd | |
| Fibrinogen | nd | | nd | | 235 | (205-249) | 151 | (101-212) |
| Saline | nd | | nd | | nd | | nd | |
| PT (%) | | | | | | | | |
| Platelet concentrate | 115 | (112-120) | 111 | (106-115) | 120 | (108-126) | 112 | (106-130) |
| Fibrinogen | 112 | (107-122) | 107 | (102-113) | 104 | (94-112) | 98 | (86-102) |
| Saline | 113 | (106-121) | 103 | (99-106) | 99 | (96-114) | 106 | (98-118) |
| aPTT(%) | | | | | | | | |
| Platelet concentrate | 31 | (28-32) | 29 | (24-34) | 27 | (22-29) | 27 | (24-28) |
| Fibrinogen | 28 | (23-30) | 28 | (24-37) | 31 | (25-37) | 31 | (26-38) |
| Saline | 31 | (23-32) | 28 | (25-34) | 28 | (24-30) | 30 | (23-32) |
| AT (%) | | | | | | | | |
| Platelet concentrate | 93 | (91-99) | 87 | (74-92) | 92 | (86-97)^{Δ} | 73 | (68-82)^{Δ} |
| Fibrinogen | 97 | (91-101) | 84 | (81-94) | 80 | (76-82)* | 61 | (45-70)^{#} |
| Saline | 106 | (94-108) | 85 | (78-91) | 80 | (77-82) | 72 | (64-77) |
| D-dimer (μg L⁻¹) | | | | | | | | |
| Platelet concentrate | 240 | (200-273) | 228 | (174-279) | 222 | (203-252) | 219 | (184-264) |
| Fibrinogen | 212 | (200-248) | 206 | (194-222) | 207 | (196-221) | 261 | (252-379)^{#} |
| Saline | 256 | (192-275) | 243 | (205-291) | 227 | (189-265) | 217 | (200-244) |
| TAT (μg L⁻¹) | | | | | | | | |
| Platelet concentrate | 20 | (12-24) | 39 | (18-47) | 42 | (26-120) | 62 | (34-120) |
| Fibrinogen | 20 | (14-29) | 30 | (21-53) | 40 | (19-120) | 57 | (53-101) |
| Saline | 23 | (13-31) | 25 | (19-48) | 26 | (18-57) | 37 | (29-79) |

PT, prothrombin time;
aPTT, activated partial prothrombin time;
AT, antithrombin;
TAT, thrombin-antithrombin;
nd, not detectable.
*P < 0.05 fibrinogen group vs. platelet group;
^{#}P < 0.05 fibrinogen group vs. saline group;
^{Δ}P < 0.05 platelet group vs. saline group for comparison of calculated differences between measurement points 2 and 3 and 3 and 4.

1.-30. (canceled)

31. A method for treating a subject suffering from excessive bleeding in a pre-hospital setting comprising administering to the subject in need of such treatment an anti-hemorrhagic pharmaceutical composition consisting of human fibrinogen as the active ingredient, wherein administration of the pharmaceutical composition is performed in a pre-hospital setting.

32. The method of claim 31, wherein the anti-hemorrhagic pharmaceutical composition is administered prior to infusion of fluids which compensate for blood volume loss.

33. The method of claim 31, wherein the anti-hemorrhagic pharmaceutical composition is administered shortly after initiation of infusion of fluids, wherein a volume of the fluids is lower than about 500 ml.

34. The method of claim 31, wherein human fibrinogen is selected from the group consisting of fibrinogen isolated from human plasma and recombinant fibrinogen.

35. The method of claim 34, wherein the fibrinogen isolated from human plasma is fibrinogen concentrate which has undergone a plurality of viral inactivation steps.

36. The method of claim 31, wherein the excessive bleeding is due to a cause selected from the group consisting of traumatic injuries, open wounds, and internal bleeding.

37. The method of claim 31, wherein the pharmaceutical composition is administered by intravenous injection or infusion.

38. The method of claim 31, wherein human fibrinogen is present within the pharmaceutical composition in an amount ranging from about 1 g to about 10 g.

39. The method of claim 31, wherein the pharmaceutical composition is formulated in a dry form or liquid form.

40. The method of claim 39, wherein if the pharmaceutical composition is formulated in a liquid form, the volume of the pharmaceutical composition is up to 100 ml.

41. The method of claim 31, wherein the pharmaceutical composition is storage-stable at ambient temperatures

42. A method for treating excessive bleeding in a subject suffering from quantitative or qualitative platelet disorder comprising administering to the subject in need of such treatment an anti-hemorrhagic pharmaceutical composition consisting of human fibrinogen as the active ingredient, wherein human fibrinogen is selected from the group consisting of fibrinogen isolated from human plasma, and recombinant human fibrinogen.

43. The method of claim 42, wherein the quantitative platelet disorder is thrombocytopenia.

44. The method of claim 42, wherein the qualitative platelet disorder is Glanzmann's Thrombasthenia (GT).

45. The method of claim 42, wherein the qualitative platelet disorder is Bernard-Soulier disease.

46. The method of claim 42, wherein the fibrinogen isolated from human plasma is fibrinogen concentrate which has undergone a plurality of viral inactivation steps.

47. The method of claim 42, wherein administering the pharmaceutical composition is performed by intravenous injection or infusion.

48. The method of claim 42, wherein human fibrinogen is present within the pharmaceutical composition in an amount ranging from about 1 g to about 10 g.

49. The method of claim 42, wherein the pharmaceutical composition is formulated in a liquid form or dry form.

50. A method of treating an individual suffering from excessive bleeding having plasma fibrinogen levels above 1-1.5 g/L comprising administering to the subject an anti-hemorrhagic pharmaceutical composition consisting of human fibrinogen as the active ingredient in an amount ranging from about 1 g to about 10 g.
